(19) United States
(12) Patent Application Publication
(10) Pub. No.: US 2009/0312441 A1
(43) Pub. Date: Dec. 17, 2009

(54) ORGANIC COMPOUNDS

(75) Inventors: Michael Ambühl, Rheinfelden (CH); Dieter Becker, Freiburg (DE); Olivier Lambert, Spechbach-leHaut (FR); Barbara Lückel, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Vienna (AT)

(21) Appl. No.: 11/721,553

(22) PCT Filed: Dec. 20, 2005
(86) PCT No.: PCT/EP05/13705

§ 371 Date: Aug. 5, 2008

(30) Foreign Application Priority Data
Dec. 22, 2004 (GB) 0428152.3

(51) Int. Cl. A61K 47/32; A61K 47/36; A61K 47/38

(52) U.S. Cl. 514/772.5; 514/772.6; 514/778; 514/779; 514/781

(57) ABSTRACT

A solid pharmaceutical composition comprising a poorly water soluble drug, a polymer which is solid at room temperature, and a disintegrant in an amount of less than 10% by weight, wherein the amounts by weight are based on the total weight of the composition.

The present invention relates to galenic compositions, in particular galenic compositions comprising a poorly water-soluble drug.

Poorly water soluble drugs are usually characterized by specific difficulties in relation to galenic compositions and by low absorption and poor bloavailability. The bioavailability of poorly soluble drugs is limited by their solubility and dissolution rates.

In order to meet these and related difficulties U.S. Pat. No. 6,350,786 describes compositions comprising stable water-insoluble complexes of poorly soluble compounds molecularly dispersed in water insoluble ionic polymers. WO 01/47492 provides a composition in the form of a solid dispersion of the practically insoluble drug and a polymer having acidic functional groups, which composition forms a suspension in vitro. EP 904060 provides particles consisting of a solid dispersion comprising itracanazole, a poorly soluble antifungal, and a water soluble polymer, preferably hydroxypropylmethylcellulose acetate succinate (HPMCAS).

WO 02/089773 provides a solid pharmaceutical composition comprising a poorly water soluble drug, a polymer which is solid at room temperature, and a surfactant which is solid at room temperature and which has a HLB value of between 8 and 17.

Polymers typically used in the manufacture of solid dispersions of poorly soluble drugs require high amounts of disintegrants in the final tablet in order to achieve an acceptable dissolution rate. This reduces the amount of solid dispersion that can be incorporated into the tablet and thus the final dose per tablet. Another difficulty in using high amounts of disintegrants is that the susceptibility of the tablet to water sorption is increased thus leading to stability problems.

In accordance with the present invention it has now surprisingly been found that particularly suitable galenic compositions comprising poorly water soluble drugs having fast dissolution rates, and interesting bioavailability characteristics are obtainable using a solid polymer which is solid at room temperature and a disintegrant in an amount of less than 10%, e.g. 5%, e.g. 2.5% by weight.

The present invention provides in one aspect a solid pharmaceutical composition, e.g. in form of a tablet or powder, comprising
(i) a poorly water soluble drug
(ii) a polymer which is solid at room temperature
(iii) a disintegrant in an amount of less than 10% by weight wherein the amount is based on the total weight of the composition.

The poorly water soluble drug is for example a lipophilic drug. The term “poorly water soluble”, is to be understood as defined in the United States Pharmacopeia, USP 26, 2003, p 2546 i.e. a “very slightly soluble” compound requiring from 1000 to 10000 parts of solvent for 1 part of solute; a “practically insoluble” or “insoluble” compound requiring more than 10000 parts of solvent for 1 part of solute.

Examples of therapeutic classes of poorly soluble drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol reducing agents, antiobesity agents, autoimmune disorder agents, anti-impotence agents, antibacterial and antifungal agents, hypnotic agents, antibiotics, anti-depressants, anti-Parkinsonism agents; anti-Alzheimer's disease agents; antiviral agents, immunosuppressants and combinations of the foregoing.

Suitable poorly water-soluble drugs of the present invention may be cyclosporins e.g. any of those having pharmaceutical utility, e.g. as immunosuppressive agents, anti-parasitic agents and agents for the reversal of multi-drug resistance, e.g. as known and described in the art, in particular Cyclosporin A (also known as Ciclosporin), Cyclosporin G, [O-(2-hydroxyethyl)-(D)Ser]⁸-Ciclosporin, and [3′-dehydroxy-3′-keto-MeBmt]¹-[Val]²-Ciclosporin. Cyclosporin A is preferred.

According to the present invention suitably polymers which are solid at room temperature are used The polymer preferably is a hydrophilic polymer. One or more polymers may be used.

Suitable pH-dependent polymers include:

Cellulose derivatives such as hydroxypropylmethylcellulose phthalate, hydroxypropylmethyl-cellulose acetate succinate or cellulose acetate phthalate. For example, hydroxypropylmethylcellulose acetate succinate (HPMCAS) may be used as known and commercially available, e.g. from Shin-Etsu. Preferably, cellulose acetate phthalate may be used as known and commercially available, e.g. from Eastman Chemical Company, US, under the trade name C-A-P (Fiedler loc. cit. p. 363). For example, hydroxypropylmethylcellulose phthalate may be used as known and commercially available, e.g. from Shin-Etsu, under the name HPMCP HP50, having a viscosity of 190±20 mPa s, a methoxy content of 20.0-25.0%, hydroxypropyl content of 5.0-10.0%, and a carboxybenzoyl content of 20.0-24.0%, or HPMCP HP55, having a viscosity of 240±20 mPa s, a methoxy content of 18.0-22.0%, hydroxypropyl content of 4.0-9.0%, and a carboxybenzoyl content of 27.0-35.0% (Fiedler, loc. cit., p. 866).

Any one or several of the polymers specified above may be used in the composition of the invention. For example, hydroxypropylmethylcellulose phthalate, as known and commercially available, e.g. from Shin-Etsu, under the name HPMCP HP50 (Handbook of Pharmaceutical Excipients loc. cit. p. 301) may be used.

In a further aspect of the invention there is provided a solid dispersion comprising a poorly soluble drug and a polymer selected from cellulose derivatives, e.g. hydroxypropylmethyl-cellulose phthalate.

In yet a further aspect there is provided a solid dispersion as hereinabove described in combination with a disintegrant in an amount of less than 10% by weight. For example, in one aspect there is provided a solid oral dosage form, e.g. tablet, comprising an inner phase comprising the solid dispersion comprising a polymer as hereinabove described, e.g. hydroxypropylmethyl-cellulose phthalate, and an outer phase comprising a disintegrant in an amount of less than 10% by weight.

In the pharmaceutical compositions of the invention the constitutional ratio of poorly water-soluble drug:polymer may be from about (10 to 70): (90 to 30), e.g. 10:90, 20:80, 30:70, 50:50, 60:40, or 70:30.

The term “disintegrant” as used herein is understood to mean a substance or mixture of substances which facilitates disintegration of the composition after administration in order that the active ingredient be released from the composition as efficiently as possible to allow for its rapid dissolution (see e.g. “Remington's Pharmaceutical Science” 18th edition (1990), “The Theory and Practice of Industrial Pharmacy” Lachman et al. Lea & Febiger (1970)).

Suitable disintegrants include but are not limited to: natural starches, such as maize starch, potato starch, and the like, directly compressible starches, e.g. Sta-rx® 1500, modified starches, e.g. carboxymethyl starches and sodium starch glycolate, available as Primojel®, Explotab®, and starch derivatives such as amylase;

Crosslinked polyvinylpyrrolidones, e.g. crospovidones, e.g. Polyplasdone® XL and Kollidon® CL;

Alginic acid or sodium alginate;

Methacrylic acid-divinylbenzene copolymer salts, e.g. Amberlite® IRP-88, and

Cross-linked sodium carboxymethylcellulose, available as e.g. Ac-di-sol®, Primellos®, Pharmacel® XL, Exlplocel, and Nymcel® ZSX.

Any one or several of the above disintegrants may be used. A combination of disintegrants may also be used. A preferred disintegrant may be crospovidone e.g. as known and commercially available under the trade name Polyplasdone XL (Fiedler loc.cit p. 1377) and Kollidon CL (Fiedler loc.cit. p. 977).

The disintegrant may be present in an amount of less than 10%, e.g. less than 5%, e.g. at or less than 2.5% by weight based on the total weight of the composition.

The solid pharmaceutical composition of the invention may further comprise a surfactant. The surfactant is for example one which can exist in the form of a, e.g. flowable, powder, having a melting point of e.g. above 40° C. The surfactant is for example nonionic, ionic or amphoteric surfactant. Surfactants may have solubilizing power for the poorly water-soluble drug. One or a mixture of surfactants may be used.

Surfactants of the non-ionic type include:

Polyoxyethylene alkyl ethers; preferably the alkyl ethers are of C₁₂ to C₁₈ alcohols. For example the polymer number is from about 2 to about 150, e.g. about 5 to about 150. For example the polymers are polyoxyethylene glycol ethers, for example polyoxyl 2-, 1 or 20-cetyl ether or polyoxyl 23lauryl ether, or polyoxyl 20-oleyl ether, or polyoxyl 2-, 10-, 20- or 100-stearyl ether, as known and commercially available e.g. under the trade mark Brij® from Uniqema. A product of this class is e.g. Brij® 35 (polyoxyl 23 lauryl ether), Brij® 58, Brij® 78P (polyoxyl 20 stearyl ether), or Brij® 98 (polyoxyl 20 oleyl ether) and polyethoxylated (20) cetyl ether, e.g. Nikkol® BC-20 TX, (Fiedler, loc. cit., pp. 326 and “Handbook of Pharmaceutical Excipients” loc. cit. p. 469).

Similar products which may also be used are polyoxyethylene-polyoxypropylene-alkyl ethers, e.g. polyoxyethylene-polyoxypropylene-ethers of C₁₂ to C₁₈ alcohols, e.g. polyoxyethylen-20-polyoxypropylene-4-cetylether which is known and commercially available under the trade mark Nikkol PBC® 34, from e.g. Nikko Chemicals Co., Ltd.

Polyethoxylated fatty acid esters with a molecular weight from about 600 to about 18 000 Daltons. For example the polymerization number is from about 8 to about 400. For example the fatty acid is of 12 to 20 carbon atoms, e.g. stearic acid, e.g. of the type known and commercially available under the trade name Myrj® from Uniqema (Fiedler, loc. cit., p. 1166). An especially preferred product of this class is Myrj® 52 having a D25 of about 1.1, a melting point of about 40 to 44° C., an HLB value of about 16.9, an acid value of about 0 to 1 and a saponification no. of about 25 to 35, or Myrj® 53, or Myrj® 59 (polyethyleneglycol-100-stearate), e.g. from Uniqema.

Polyethoxylated sorbitan monostearates, e.g. as known and commercially available under the trade name Tween® 61 from Uniqema (Fiedler, loc. cit., pp. 1754).

Polyethoxylated distearates, e.g. as known and commercially available under the trade name Atlas® G 1821 from Uniqema (Fiedler, loc. cit., vol. 2, pp. 264), or Nikko® CDS-6000P from Nikko Chemicals Co., Ltd.

Polyoxyethylene(POE)-polyoxypropylene(POP)-polyoxyethylene(POE) surfactants, e.g. poloxamers, e.g. poloxamer 188, as known and commercially available under the tradename of Pluronic® F 68 from BASF (Handbook of Pharmaceutical Excipient, loc.cit., p. 449) or Synperonic® PE/F 68 from Uniqema, or e.g. poloxamer 407 as known and commercially available under tradename Pluronic® F 127 from BASF or Synperonic PE/F 127 from Uniqema.

Vitamin E based surfactants, e.g. as known and commercially available under the name Vitamin E TPGS (polyethoxylated tocopherol succinate) from e.g. Eastman Kodak (Handbook of Pharmaceutical Excipients, loc. cit., p. 27).

Sucrose esters, e.g. sucrose stearate or sucrose palmitate.

Monoglyceride based food emulsifiers, e.g. as known and commercially available under the trade name Panodan® AM VEG from Danisco (Fiedler, loc. cit., p. 1264), or citric acid esters of monoglyceride, e.g. Citrem® LC VEG from Danisco.

Polyethoxylated hydrogenated castor oil, e.g. as known and commercially available under the trade name Cremophor® RH 60 from BASF (Fiedler, loc. cit., p. 474), which has a saponification value of about 40 to 50, an acid value less than about 1, an iodine value of less than about 1, a water content (Fischer) of about 4.5 to 5.5%, an n_{D}⁶⁰ of about 1.453 to 1.457 and an HLB of about 15 to 17.

Polyethylene glycol (PEG) sterol ethers having, e.g. from 5 to 35 [CH₂—CH₂—O] units, e.g. 20 to 30 units, also in combination with polyoxethylene alkyl ethers. Preferably the polymer is as known and commercially available under the trade name Solulan® C24 (Choleth 24 (and) Ceteth 24) from Amerchol (Fiedler, loc. cit., p. 1555), or Forlan® C-24 (Choleth 24 (and) Ceteth 24) from R.I.T.A. Corp. (Fiedler, loc. cit., p. 744)

Similar products which may also be used are those which are known and commercially available under the trade name Nikkol® BPS-30 (polyethoxylated 30 phytosterol) or Nikkol® BPSH-25 (polyethoxylated 25 phytostanol), from e.g. Nikko Chemicals Co., Ltd.

Surfactants of the anionic type include:

Sodium alkyl sulfates e.g. sodium C₈-C₁₈alkyl sulfates, e.g. sodium C₁₀-C₁₈alkyl sulfates, e.g. sodium lauryl sulfate, which Is also known as sodium dodecyl sulfate and which is commercially available, e.g. under the trade name Texapon K12® from Henkel KGaA (Fiedler, loc. cit., pp. 1692);

Sodium alkyl sulfonates, e.g. sodium C₈-C₁₈alkyl sulfonates, e.g. sodium C₁₀-C₁₈alkyl sulfonates;

Sodium alkyl aryl sulfonates, e.g. sodium C₈-C₁₈alkyl aryl sulfonates, e.g. sodium C₁₀-C₁₈alkyl aryl sulfonates, wherein aryl is e.g. benzyl, phenyl and the like;

Sodium alkyl phosphate e.g. sodium CrC₁₈alkyl phosphate, e.g. sodium C₁₀-C₁₈alkyl phosphate, e.g. sodium lauryl phosphate, or e.g. potassium cetyl phosphate, available under the trade name of AMPHISOL K from Hoffmann La Roche Ltd.;

Sodium stearoyl lactylate (sodium-O-stearyllactate), e.g. as known and commercially available under the name SSL P55 VEG from Danisco; or

Sodium (C₄-C₁₂) fatty acid salts e.g. sodium caprinate (Fiedler, loc. cit., p. 1176).

Surfactants of the amphotheric type include:

Lecithins, e.g. soy bean phospholipid, e.g. as known and commercially available under the trade name Lipoid® S75 from Lipoid; or egg phospholipid, e.g. as known and commercially available under the trade name Phospholipon® 90 from Nattermann (Fiedler, loc. cit., vol. 2, pp. 1315)

The composition may further comprise a carrier e.g. a water-soluble or water-insoluble saccharide such as lactose or mannitol; microcrystalline cellulose, e.g. as known and commercially available under the trade name Avicel®, from FMC Corporation; A mixture of lactose and microcrystalline cellulose may be used, e.g. MicroceLac® 100, a spray-dried compound consisting of 75% α-lactose monohydrate and 25% microcrystalline cellulose produced by Meggle (Fiedler loc. cit. p. 1128).

The composition may further comprise one or more lubricants. Lubricants may be present in a total amount of up to 5% by weight, e.g. up to 2%, e.g. up to 1% by weight based on the total weight of the composition.

Examples of such lubricants include: magnesium stearate (Faci), sodium benzoate, glyceryl mono fatty acid, e.g. having a molecular weight of from 200 to 800 Daltons e.g. gylceryl monostearate (e.g., Danisco, UK), glyceryl dibehenate (e.g., CompritolATO888™, Gattefossé France), glyceryl palmito-stearic ester (e.g. Precirol™, Gattefossé France), polyoxyethylene glycol (PEG, BASF), hydrogenated cotton seed oil (Lubitrab, Edward Mendell Co Inc), castor seed oil (Cutina HR, Henkel).

The composition may further comprise one or more glidants e.g. colloidal silicon dioxide (Aerosil, Degussa). Glidants may be present in an amount of e.g. up to 2%, e.g. up to 1%.

Excipients disclosed in the literature, as for instance in Fiedler's “Lexikon der Hilfsstoffe”, 5^{th} Edition (2002), ECV Aulendorf and “Handbook of Pharmaceutical Excipients”, Rowe, Sheskey and Weller, Fourth Edition (2003), the contents of which are incorporated herein by reference, may be used in the pharmaceutical compositions according to the invention. Conveniently the excipients comprise less than 40% of the weight of the dosage form.

The compositions of the invention may be prepared by working up active agent with the excipients. The following processes A to H are contemplated.

A. In one aspect the compositions of the present invention in form of a solid dispersion comprising (1) a poorly water-soluble drug, a polymer and optionally a surfactant, may be obtained by
(i) dissolving, suspending or dispersing the drug and the polymer in a solvent or solvent mixture,
(ii) adding the surfactant if present to the drug/polymer/solvent mixture,
(iii) evaporating the solvent and co-precipitating the drug with the polymer and the surfactant if present,
(iv) drying the resulting residue, e.g. under reduced pressure, milling and sieving the particles.

The solvent of (i) may be a single solvent or a mixture of solvents. Suitable solvents for use according to the present Invention may be organic solvents such as an alcohol, e.g. methanol, ethanol, or isopropanol; an ester, e.g. ethylacetate; an ether, e.g. diethylether; a ketone, e.g. acetone; or a halogenated hydrocarbon, e.g. dichloromethane. Preferably a solvent mixture of ethanol/acetone having a weight ratio of ethanol:acetone of between about 1:10 to about 10:1, e.g. 1:5 to 5:1 may be used.

B. In another aspect the compositions of the present invention in form of a solid dispersion comprising (1) a poorly water-soluble drug, (2) a polymer and (3) a surfactant may be obtained by
(i) dissolving, suspending or dispersing the drug, and surfactant, if present, in a solvent or solvent mixture and optionally adding small amounts of water, if necessary,
(ii) adding the polymer, to the drug/solvent or drug/surfactant/solvent mixture,
(iii) evaporating the solvent and co-precipitating the drug, with the surfactant and the polymer,
(iv) drying the resulting residue, e.g. under reduced pressure, milling and sieving the particles.

The solvent of (i) may be a single solvent or a mixture of solvents. Suitable solvents for use according to the present invention may be organic solvents such as an alcohol, e.g. methanol, ethanol, or isopropanol; an ester, e.g. ethylacetate; an ether, e.g. diethylether, a ketone, e.g. acetone; or a halogenated hydrocarbon, e.g. dichloromethane. Preferably a solvent mixture of ethanol/acetone having a weight ratio of ethanol:acetone of between about 1:10 to about 10:1, e.g. 1:5 to 5:1 may be used.

C. Alternatively, the solid dispersions of the invention, comprising a poorly water-soluble drug, a polymer and/or a surfactant, may be prepared by spray-drying techniques including fluid-bed spray-drying. A solution or dispersion as formed above is dispersed through a nozzle at an inlet temperature of about 50 to about 130° C. into a chamber. The solvent is evaporated through the nozzle, and finely dispersed particles are collected.

D. In a further alternative embodiment of the present invention the solid dispersion, comprising (1) a poorly water-soluble drug, (2) a polymer and optionally (3) a surfactant, may be prepared by spraying the solution or dispersion onto (4) a carrier in the fluid-bed (i.e. spray-granulation).

The particles typically have a mean particle size of less than about 2 mm, e.g. 1 mm, e.g. 0.5 mm, as measured e.g. by light microscopy.

F. The compositions of the present invention wherein the poorly water-soluble drug, is encapsulated in a polymeric matrix, e.g. in form of microparticles, may be prepared e.g. according to a process comprising the following steps:
(i) preparation of an Internal organic phase comprising
(ia) dissolving the polymer in an organic solvent or solvent mixture. The solvent may be a single solvent or a mixture of solvents. Suitable solvents for use according to the present invention may be organic solvents such as a ketone, e.g. acetone; or a halogenated hydrocarbon, e.g. methylene chloride. Preferably a solvent mixture of methylene chloride/acetone having a weight ratio of methylene chloride:acetone of between about 1:10 to about 10:1, e.g. 1:5 to 5:1, preferably 1:1, may be used,
(ib) adding the poorly water-soluble drug, to the polymer solution, and optionally
(ic) adding a surfactant to the solution obtained by step (ib),
(ii) preparation of an external aqueous phase comprising
(iia) preparing a buffer, e.g. acetate buffer,
(iib) dissolving gelatin or polyvinylalcohol (PVA) in water, and
(iic) mixing the solution obtained by step (iib) with the solution obtained by step (iia) to obtain e.g. a 0.5% gelatin solution in the buffer,
(iii) mixing the internal organic phase, e.g. brought at 20 ml/min with a gear pump, with the external aqueous phase, e.g. brought at 400 ml/min with a gear pump, e.g. in a ratio of internal phase to external phase of about 1:10 to about 1:40, preferably about 1:20, with a device creating high shear forces, e.g. with a static mixer, to form e.g. an oil/water emulsion, and
(iv) hardening the microparticles by solvent evaporation, washing for excipients removal and collecting the microparticles.
The microparticles typically have a mean particle size of less than about 350 microns, e.g. about 1 to about 180 microns, as measured e.g. by scanning electron microscopy.
In order to e.g. increase flowability of the final microparticle powder, the obtained microparticles may be further worked up by adding an aqueous solution of a carrier, e.g. lactose, and lyophilization or spray drying of the resulting suspension to obtain a, e.g. flowable, powder.
The compositions of the invention in powder form, e.g. particles, e.g. solid dispersion particles or microparticles, may be compressed to tablets.

The particles, e.g. solid dispersion particles or microparticles, may be combined with one or more flow enhancers, e.g. colloidal silicon dioxide, and/or one or more solid surfactants as specified above, e.g. sodium lauryl sulfate, e.g. in a total amount of enhancers and/or surfactants of 0.1 to 20% by weight, e.g. 1 to 10 % by weight based on the total weight of the composition.

If present in the compositions, the filler or a mixture of fillers, the disintegrants or a mixture of disintegrants, the lubricants or a mixture of lubricants, the flow enhancers or a mixture of flow enhancers, the additional surfactant or surfactants may be added to the drug/polymer/solvent mixture, the drug/surfactant/solvent mixture, the drug/polymer/surfactant/solvent mixture or, preferably, to the outer tabletting phase.

The outer tabletting phase may comprise e.g. spray-dried lactose/microcrystalline cellulose mixtures or a mixture of α-lactose monohydrate and microcrystalline cellulose, e.g. Microcelac 100, e.g. to achieve tablet compositions with a suitable average hardness

In one embodiment, the present invention provides tablet compositions with an average hardness of e.g. from 60 to 150 N, preferably from 80 N to 110 N.

In another embodiment the present invention provides a tablet with a disintegration time of less than 6 minutes, e.g. less than 4 minutes, e.g. less than 2 minutes.

In a further embodiment the dosage form has 60 to 70 % or greater release of the poorly water soluble drug i.e. Cyclosporine A within 15 minutes, indicated in standard in vitro dissolution tests, e.g. effected by use of the apparatus 2 (Rotary Paddle) of the USP at 37 degrees Celsius in water at a stirring rate of 60 rpm using sodium dodecyl sulphate as a solubilizing agent at a concentration of 1% for 600 mg dosage form and based on the mean of 6 or more-e.g. 10 dosage forms.

Examples of minimum release rates are:
a) 62% or
b) 70%
   within 15 minutes (after the start of the experiment)

Examples of maximum release rates are:
a) 77% or
b) 81%
   within 15 minutes (after the start of the experiment)

According to the present invention there is advantageously provided a composition comprising a poorly water-soluble drug and a polymer enabling a fast disintegrating time and a fast dissolution time of the final tablet without the need of high a amounts of a disintegrant. The compositions of the invention show improved bloavailability and excellent stability. For example, solid oral dosage forms comprising the compositions of the invention may be packed without requiring a highly protective and thus expensive blister foil.

Compositions of the invention comprising a poorly soluble drug may be used alone or together with other poorly soluble drugs.

The utility of all the pharmaceutical compositions of the present invention may be observed in standard clinical tests in, e.g., known indications of drug dosages giving therapeutically effective blood levels of drug, e.g., using dosages in the range of 2.5-1000 mg of drug per day for a 75 kg mammal, e.g., adult and in standard animal models. The increased bioavailability of the drug provided by the compositions may be observed in standard animal tests and in clinical trials, e.g., as known in the art.

Following is a non-limiting description by way of examples:

### EXAMPLE 1

Compositions of Examples 1 and 1A are made by dissolving Cyclosporin A in an ethanol/acetone mixture, adding the polymer, surfactant, and carrier medium, mixing until homogenously dispersed, evaporation of the solvents, and drying, milling and sieving the resulting residue. The resulting particles are mixed with the additional excipients and directly compressed to flat tablets.

| Component | Example 1 | Example 1A |
|---|---|---|
| CsA | 16.7 | 16.7 |
| HPMCP | 22.9 | 22.9 |
| Myrj 59 | 2.1 | 2.1 |
| Microcelac (75% alpha-lactose | 55 | 37.5 |
| monohydrate and | | |
| 25% microcrystalline cellulose) | | |
| Crospovidone | 2.5 | 20 |
| Mg-stearat | 0.5 | 0.5 |
| Aerosil | 0.3 | 0.3 |
| Tablet weight | 300 mg | 300 mg |
| Hardness N | 93 | 91 |
| Disintegration time | 1′00-1′30 min. | 20-23 sec. |
| Dissolution rate/15 min | 77.4 ± 4.2 | 80.6 ± 8.6 |

Comparative Example 1A indicates that according to the present invention low amounts of disintegrant (example 1) at a comparable hardness of the tablet still achieve a disintegration time less than 1′30 min with a comparable dissolution rate.

1. A solid pharmaceutical composition comprising
a poorly water soluble drug,
a polymer which is solid at room temperature, and
a disintegrant in an amount of less than 10% by weight,
wherein the amounts by weight are based on the total weight of the composition.

2. The composition as claimed in claim 1 wherein the disintegrant is present in less than 5% by weight, wherein the amounts by weight are based on the total weight of the composition.

3. The composition as claimed in claim 1 wherein the disintegrant is present at about or in less than 2.5% by weight, wherein the amounts by weight are based on the total weight of the composition.

4. The composition as claimed in claim 1 wherein the disintegrant is selected from natural starches, modified starches, starch derivatives, crosslinked polyvinylpyrrolidones, alginic acid, sodium alginate, methacrylic acid-divinylbenzene copolymer salts and cross-linked sodium carboxymethylcellulose.

5. The composition as claimed in claim 1 wherein the disintegrant is crospovidone.

6. The composition as claimed in claim 1 wherein the polymer is selected from cellulose derivatives.

7. The composition as claimed in claim 6 wherein the cellulose derivatives are hydroxypropylmethylcellulose phthalate, hydroxypropylmethylcellulose acetate succinate or cellulose acetate phthalate.

8. The composition as claimed in claim 1 further comprising a lubricant.

9. The composition as claimed in claim 1 further comprising a glidant.

10. The composition as claimed in claim 1 further comprising a carrier.

11. The composition as claimed in claim 10 wherein the carrier is saccharide or microcrystalline cellulose.

12. The composition as claimed in claim 10 wherein the carrier further comprises lactose.

13. The composition as claimed in any preceding claim 1 wherein the composition is in form of a solid dispersion.

14. A solid dispersion comprising a poorly soluble drug and a polymer selected from cellulose derivatives.

15. A solid dispersion of claim 14 wherein the polymer is hydroxypropylmethylcellulose phthalate.

16. A solid dispersion of claim 14 in combination with a disintegrant in an amount of less than 10% by weight.

17. A solid oral dosage form comprising an inner phase comprising the solid dispersion of claim 14 and an outer phase comprising a disintegrant in an amount of less than 10% by weight.

18. A package comprising a composition as claimed in any one of claims 1 or a solid oral dosage form as claimed in claim 17 and instructions to use.

19. A process for the preparation of the composition of claim 1 comprising the steps
(i) dissolving, suspending or dispersing a poorly water soluble drug and a polymer in a solvent or solvent mixture,
(ii) otionally adding a surfactant to the drug/polymer/solvent mixture,
(iii) evaporating the solvent and co-precipitating the poorly water soluble drug with the polymer and the surfactant,
(iv) drying the resulting residue under reduced pressure, milling and sieving the particles.

20. A process for the preparation of the composition of claim 1 comprising the steps
(i) dissolving, suspending or dispersing a poorly water soluble drug and a polymer in a solvent or solvent mixture,
(ii) optionally adding a surfactant to the drug/polymer/solvent mixture,
(iii) dispersing the mixture through a nozzle at an inlet temperature of about 50 to about 130° C. into a chamber,
(iv) evaporating the solvent through the nozzle, and
(v) collecting the dispersed particles

21. A process for the preparation of the composition of claim 1 comprising the steps
(i) dissolving, suspending or dispersing a poorly water soluble drug and a polymer in a solvent or solvent mixture,
(ii) otionally adding a surfactant to the drug/polymer/solvent mixture,
(iii) spraying the solution or dispersion onto a carrier in a fluid-bed, and
(iv) Collecting the spray-granulated particles
